(19) United States
(12) Patent Application Publication
(10) Pub. No.: US 2020/0163856 A1
(43) Pub. Date: May 28, 2020

(54) SUNSCREEN AGENT WITH PROTECTION AGAINST TEXTILE SPOTTING DUE TO 2,4-BIS-{[4-(2-ETHYL-HEXYLOXY)-2-HYDROXY]-PHENYL}-6-(4-METHOXYPHENYL)-1,3,5-TRIAZINE

(71) Applicant: BEIERSDORF AG, Hamburg (DE)

(72) Inventors: Katrin RESKE, Buxtehude (DE); Toshihiko SHIMODA, Tokyo (JP); Janina ZIPPEL, Hamburg (DE); Andreas BLECKMANN, Ahrensburg (DE); Doreen GUTZKE, Bispingen (DE)

(21) Appl. No.: 16/629,635

(22) PCT Filed: Jun. 29, 2018
(86) PCT No.: PCT/EP2018/067604

§ 371 Date: Jan. 9, 2020

(30) Foreign Application Priority Data
Jul. 13, 2017 (DE) 102017212017.7

(51) Int. Cl. A61K 8/49; A61Q 17/04; A61K 8/06; A61K 8/11

(52) U.S. Cl. CPC A61K 8/4966; A61Q 17/04; A61K 8/06; A61K 2800/592; A61K 2800/30; A61K 2800/624; A61K 8/11

(57) ABSTRACT

The invention relates to a cosmetic preparation containing 2,4-Bis-{[4-(2-ethyl-hexyloxy)-2-hydroxy]-phenyl}-6-(4-methoxyphenyl)-1,3,5-triazine (inci: Bis-Ethylhexyloxyphenol Methoxyphenyl Triazine), which is encapsulated with cellulose acetate, and to methods and to uses of 2,4-Bis-{[4-(2-ethyl-hexyloxy)-2-hydroxy]-phenyl}-6-(4-methoxyphenyl)-,3,5-triazine (INCI: Bis-Ethylhexyloxyphenol Methoxyphenyl Triazine) which is encapsulated with cellulose acetate.

The present invention relates to a cosmetic preparation containing 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate and also to processes and uses of 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate.

There has been a continuing trend away from pale complexion towards “healthy, athletic tanned skin” for many years. To achieve this, people expose their skin to sunlight, since this causes pigment formation in the sense of melanin formation. However, the ultraviolet radiation of sunlight also has a harmful effect on skin. In addition to acute damage (sunburn), long-term damage such as an increased risk of developing skin cancer occurs on excessive irradiation with light from the UVB range (wavelength: 280-320 nm). Excessive exposure to UVB and UVA radiation (wavelength: 320-400 nm) additionally leads to a weakening of the elastic and collagen fibers in connective tissue. This leads to numerous phototoxic and photoallergic reactions and results in premature aging of the skin.

To protect the skin, a number of photoprotective filter substances have accordingly been developed that may be used in cosmetic preparations. These UVA and UVB filters are in most industrialized countries collated in the form of positive lists such as Annex 7 of the Kosmetikverordnung [German Cosmetics Ordinance]

The plethora of commercially available sunscreens must not however obscure the fact that these prior art preparations have a number of disadvantages.

Cosmetic preparations such as sunscreen preparations that are applied to the skin regularly come into contact (intentionally or unintentionally) with clothing and linen (for example towels) and adhere to the latter to some degree (for example as a consequence of being rubbed off or of being soaked up by the fibrous material). Depending on the nature of the constituents, this results in the formation of stains and discolorations, particularly on light-colored textiles. These discolorations are caused in particular by non-water-soluble UVA and broad-spectrum filters such as 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine). The stains are difficult to remove by washing with conventional detergents and in fact intensify further during the washing process by interactions with ions in the washing water.

The object of the present invention was therefore to eliminate the disadvantages of the prior art and to develop a cosmetic preparation (in particular sunscreen) containing a non-water-soluble broad-spectrum filter such as that 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) can be washed more easily out of textiles contaminated with the preparation.

The object is surprisingly achieved by a cosmetic preparation containing 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate.

The object is further achieved by a process for facilitating the washability of cosmetic preparations comprising 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) out of textiles, characterized in that the cosmetic preparation contains 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate.

Last, but not least, the object is achieved by the use of 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate in cosmetic preparations for facilitating the washability of filters protecting against UV light out of textiles contaminated with the preparations.

Although UV filters encapsulated with polymethyl methacrylate (PMMA) are known from the prior art (for example DE102014206147, DE102014206156, DE102014206152), these documents did not point the way to the present invention.

In the context of the present disclosure, the phrases “according to the invention”, “preparation according to the invention”, etc. refer in all cases to the preparations, processes, and uses according to the invention, i.e. also to preparations in which the uses according to the invention are executed and to preparations with which the process according to the invention is executed.

One way of preparing the 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) according to the invention encapsulated with cellulose acetate is in analogous manner to example 2 of WO2017/037716, but using Bis-Ethylhexyloxyphenol Methoxyphenyl Triazine instead of titanium dioxide and bismuth oxychloride. Similarly, it can be prepared in analogous manner to example 2 of WO2014/133261, but using cellulose acetate instead of PMMA and Bis-Ethylhexyloxyphenol Methoxyphenyl Triazine instead of 4-tert.-butyl-4′-methoxydibenzoylmethane. The product can be obtained from Tagra Biotechnologies LTD, Netanya.

It is advantageous in accordance with the invention if the preparation according to the invention contains one or more further UV filters selected from the group of the compounds 4-(tert-butyl)-4′-methoxydibenzoylmethane, hexyl 2-[4-(diethylamino)-2-hydroxybenzoyl]benzoate (INCI: Diethylamino Hydroxybenzoyl Hexyl Benzoate), salts of 2-phenylbenzimidazole-5-sulfonic acid, 2,4,6-tris[anilino-(p-carbo-2′-ethyl-1′-hexyloxy)]-1,3,5-triazine (INCI: Ethylhexyl Triazone), 4,4′-[[6-[[4-[[(1,1-dimethylethyl)amino]carbonyl]phenyl]amino]-1,3,5-triazine-2,4-diyl]diimino]bis,bis(2-ethylhexyl)benzoate (INCI: Diethylhexyl Butamido Triazone), 2,4,6-tribiphenyl-4-yl-1,3,5-triazine (INCI: Tris-Biphenyl Triazine), 2-ethylhexyl 2-hydroxybenzoate (INCI: Ethylhexyl Salicylate), 3,3,5-trimethylcyclohexyl 2-hydroxybenzoate (INCI: Homosalate), 2,2′-methylenebis(6-(2H-benzotriazol-2-yl)-4-(1,1,3,3-tetramethylbutyl)phenol), 2-ethylhexyl 2-cyano-3,3-diphenylacrylate, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI: Drometrizole Trisiloxane), [(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI: Terephthalylidene Dicamphor Sulfonic Acid), merocyanine, titanium dioxide, zinc oxide.

These further UV filters may be used advantageously at individual concentrations of 0.1 to 10% by weight based on the total weight of the preparation.

In this case, it is preferable in accordance with the invention if the preparation according to the invention is free of 2-hydroxy-4-methoxybenzophenone, 2-ethylhexyl 4-methoxycinnamate, isoamyl 4-methoxycinnamate, and/or 3-(4-methylbenzylidene)camphor. Particularly preferably in this case, the use of 2-ethylhexyl 4-methoxycinnamate, isoamyl 4-methoxycinnamate, 3-(4-methylbenzylidene)camphor, and 2-hydroxy-4-methoxybenzophenone should be avoided.

In an alternative embodiment that is advantageous in accordance with the invention, the preparation according to the invention is additionally free of 2-ethylhexyl 2-cyano-3,3-diphenylacrylate (INCI: Octocrylene).

It is advantageous in the context of the present invention if the preparation contains 2,4-bis{[4-(2-ethylhexyloxy)-2-hydroxy]-phenyl}-6-(4-methoxyphenyl)-1,3,5-triazine (INCI: Bis-Ethylhexyloxyphenol Methoxyphenyl Triazine) encapsulated with cellulose acetate in an amount of 0.1 to 15% by weight, preferably in an amount of 0.5 to 10% by weight, and more preferably in an amount of 1 to 7% by weight, in each case based on the total weight of the preparation.

According to the invention, the weight ratio of cellulose acetate to Bis-Ethylhexyloxyphenol Methoxyphenyl Triazine in the capsules is advantageously 20:80 to 60:40, and preferably in accordance with the invention 30:70 to 50:50 (cellulose acetate to Bis-Ethylhexyloxyphenol Methoxyphenyl Triazine).

It is advantageous in accordance with the invention if the preparation according to the invention is in the form of an emulsion. In this case, it is preferable in accordance with the invention if the preparation is in the form of an oil-in-water emulsion (O/W emulsion). It is particularly preferred in accordance with the invention that the O/W emulsion is in the form of a lotion.

If the preparation according to the invention is in the form of an emulsion, the embodiments that are advantageous in accordance with the invention are characterized in that the preparation contains glyceryl stearate citrate, cetearyl alcohol, sodium cetearyl sulfate, glyceryl stearate, cetearyl sulfosuccinate, sodium stearoyl glutamate, polyglyceryl-3-methylglucose distearate, stearic acid, potassium cetyl phosphate, polyglyceryl-10 stearate (INCI Polyglyceryl-10 Stearate), and/or polyglyceryl-2 caprate.

These constituents are advantageously used in individual concentrations of 0.1 to 7% by weight based on the total weight of the preparation.

It is advantageous in accordance with the invention if the preparation according to the invention is free of polyethylene glycol, polyethylene glycol ethers, and polyethylene glycol esters (so-called PEG derivatives).

Embodiments of the present invention that are advantageous in accordance with the invention are characterized in that the preparation contains ethylhexylglycerol, propylene glycol, butylene glycol, 2-methylpropane-1,3-diol, 1,2-pentanediol, 1,2-hexanediol, 1,2-octanediol, and/or 1,2-decanediol.

These compounds may be present individually or in combination in the preparation according to the invention. The use concentration (individual concentration) according to the invention for these constituents is from 0.01 to 5% by weight based on the total weight of the preparation.

The use of ethylhexylglycerol is particularly advantageous in accordance with the invention. The use concentration for ethylhexylglycerol which is advantageous in accordance with the invention is from 0.1 to 1% by weight based on the total weight of the preparation.

It is advantageous in accordance with the invention if the preparation according to the invention contains phenoxyethanol, 4-hydroxyacetophenone, and/or ethanol.

Phenoxyethanol may be used advantageously in accordance with the invention at a concentration of 0.1 to 0.9% by weight based on the total weight of the preparation.

4-Hydroxyacetophenone may be used advantageously in accordance with the invention at a concentration of up to 0.4% by weight based on the total weight of the preparation.

Ethanol may be used advantageously in accordance with the invention at a concentration of up to 10% by weight based on the total weight of the preparation.

Preference is given in accordance with the invention to using a combination of phenoxyethanol and ethylhexylglycerol.

Embodiments that are preferred in accordance with the invention are obtained if the preparation does not contain parabens (particularly propylparaben and butylparaben), nor any 3-iodo-2-propynyl butylcarbamate, methylisothiazolinone, chloromethylisothiazolinone, and DMDM hydantoin, i.e. it is free of these constituents.

It is advantageous in accordance with the invention if the preparation according to the invention contains one or more perfumes selected from the list of the compounds limonene, citral, linalool, alpha-isomethyl ionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methyl ionone, amyl C, butylphenyl methylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, ethylene brassylate, Evernia furfuracea extract, Evernia prunastri extract, farnesol, guaiac wood oil, geraniol, hexylcinnamal, hexyl salicylate, hydroxycitronellal, hydroxyisohexyl 3-cyclohexenecarboxaldehyde, lavender oil, lime oil, linalyl acetate, mandarin oil, menthyl PCA, methylheptenone, nutmeg oil, rosemary oil, sweet orange oil, terpineol, tonka bean oil, triethyl citrate, and/or vanillin.

Embodiments of the present invention that are advantageous in accordance with the invention are characterized in that the preparation contains one or more oils selected from the group of the compounds butylene glycol dicaprylate/dicaprate, phenethyl benzoate, C12-15 alkyl benzoate, dibutyl adipate, diisopropyl sebacate, dicaprylyl carbonate, di-C12-13 alkyl tartrate, butyloctyl salicylate, diethylhexyl syringylidenemalonate, hydrogenated castor oil dimerate, triheptanoin, C12-13 alkyl lactate, C16-17 alkyl benzoate, propylheptyl caprylate, caprylic/capric triglyceride, diethylhexyl 2,6-naphthalate, octyldodecanol, caprylic/capric triglyceride, ethylhexyl cocoate.

These may be used in individual concentrations of 0.1 to 15% by weight based on the total weight of the preparation.

Embodiments of the present invention that are advantageous in accordance with the invention are also characterized in that the preparation contains one or more compounds selected from the group of the compounds alpha-lipoic acid, folic acid, phytoene, D-biotin, coenzyme Q10, alpha-glucosylrutin, carnitine, carnosine, glycyrrhetinic acid, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, glycerylglucose, (2-hydroxyethyl)urea, vitamin E and derivatives thereof, hyaluronic acid and/or salts thereof, and/or licochalcone A.

Embodiments of the present invention that are advantageous in accordance with the invention are further characterized in that the preparation contains Acrylate/C10-30 Alkyl Acrylate Crosspolymer and/or triacontanyl PVP.

Last but not least, it is advantageous in accordance with the invention if the preparation according to the invention contains xanthan gum, tapioca starch, and/or carboxymethyl cellulose.

It is also advantageous in accordance with the invention if the preparation according to the invention contains silica dimethyl silylate and/or talc.

The preparation according to the invention may advantageously contain moisturizers. Moisturizers are substances or mixtures of substances that give cosmetic preparations the property, when applied or spread on the surface of the skin, of reducing the loss of moisture from the stratum corneum (also known as transepidermal water loss (TEWL)) and/or of having a beneficial effect on the hydration of the stratum corneum.

The water phase of the preparations according to the invention may advantageously contain customary cosmetic auxiliaries such as alcohols, in particular those having a low number of carbon atoms, preferably ethanol and/or isopropanol or polyols having a low number of carbon atoms and also the ethers thereof, electrolytes, self-tanning agents, and in particular one or more thickeners, which may advantageously be selected from the group consisting of silica, aluminum silicates, polysaccharides and derivatives thereof, for example hyaluronic acid, xanthan gum, hydroxypropyl methylcellulose, and/or polyacrylates (preferably a polyacrylate from the group of so-called Carbopols, for example Carbopols of types 980, 981, 1382, 2984, 5984, each individually or in combination).

Measurement of Washability

The washability of the sunscreen out of textiles may be determined, for example, with the following method:

Sunscreen emulsions are investigated in respect of the formation of yellow stains on cotton textiles by means of an in-vitro application/wash cycle. White prewashed cotton monitors (100% cotton) are used for this purpose. In the test, 3 mg/cm2 of the test formulation was distributed uniformly on PMMA Schönberg plates (5.0×5.0 cm) and transferred directly to the test textile by applying pressure. The stained cotton samples are then dried in air under laboratory conditions for 12 h.

After drying, colorimetric characterization of the resulting initial staining was carried out by measuring the degree of yellowing using a Datacolor 800 colorimeter (Datacolor International).

Measurement geometry: d/8°, SCE (specular component excluded)

Illuminant/observer: D65/10° (corresponding to average daylight)

UV filtering: adjusted to D65, Ganz/Griesser method

Measurement aperture: LAV (30 mm diameter)

Sample background: substrate paper without optical brightener, test climate: 21° C. (±1° C.), 41% (±4%) rel. atmospheric humidity.

The evaluation is based on the change in the b value from the CIELAB color measurement system. The B axis in the CIELAB system characterizes the yellow/blue color impression, with positive b values representing an increase in the yellow component. The higher the b value, the greater the yellow impression. After the measurement procedure, the test cloths are washed separately in a washing machine (60° C., 2 h, Ariel Compact powder detergent, loading otherwise clean). After drying for 12 h under laboratory conditions, colorimetric characterization of the resulting staining was repeated by measuring the color values using the Datacolor 800 colorimeter (Datacolor International) as already described.

The CIELAB system or L*a*b* color space is a three-dimensional measurement space that includes all perceptible colors. The color space is constructed on the basis of opponent color theory. One of the most important properties of the L*a*b* color model is its instrument independence, i.e. the colors are defined independently of the nature of their generation and the display technology. The corresponding EU guideline is DIN EN ISO 11664-4 “Colorimetry—Part 4: CIE 1976 L*a*b* Colour space”. The coordinates of the CIELAB plane are formed from the red/green value a and the yellow/blue value b. The lightness axis L is perpendicular to this plane. According to DIN 6174, L, a and b should be written with an asterisk (*) in order to distinguish them from other systems, for example the “HunterLab” system.

### EXAMPLES

The examples below are intended to illustrate the present invention without limiting it. Unless otherwise stated, all quantitative data, fractions, and percentages are based on the weight and the total amount or on the total weight of the preparations.

| INCI | | Ex. 1 | | Ex. 2 | | Ex. 3 | |
|---|---|---|---|---|---|---|---|
| Encapsulated | Bis-Ethylhexyloxyphenol | 6.66 | 4.00 | 3.34 | 2.00 | 1.69 | 1.00 |
| anisotriazine | Methoxyphenyl Triazine | | | | | | |
| | Cellulose Acetate | | 2.66 | | 1.34 | | 0.69 |

| | | |
|---|---|---|
| Bis-Ethylhexyloxyphenol Methoxyphenyl Triazine | 2.00 | 3.00 |

| | | | |
|---|---|---|---|
| Butyl Methoxydibenzoylmethane | 4.75 | 4.75 | 4.75 |
| mosalate | 9.00 | 9.00 | 9.00 |
| Ethylhexyl Salicylate | 4.75 | 4.75 | 4.75 |
| Ethylhexyl Triazone | 3.00 | 3.00 | 3.00 |
| Titanium Dioxide (nano) | 0.82 | 0.82 | 0.82 |
| Silica | 0.15 | 0.15 | 0.15 |
| Dimethicone | 0.04 | 0.04 | 0.04 |
| Phenylbenzimidazole Sulfonic Acid | 1.00 | 1.00 | 1.00 |
| Tocopheryl Acetate | 0.06 | 0.06 | 0.06 |
| Hydroxyacetophenone | 0.40 | 0.40 | 0.40 |
| Panthenol | 1.05 | 1.05 | 1.05 |
| Ethylhexylglycerol | 0.30 | 0.30 | 0.30 |
| C12-15 Alkyl Benzoate | 2.00 | 2.00 | 2.00 |
| Butylene Glycol Dicaprylate/Dicaprate | 2.00 | 2.00 | 2.00 |
| C18-38 Alkyl Hydroxystearoyl Stearate | 1.00 | 1.00 | 1.00 |
| Glyceryl Stearate | 1.00 | 1.00 | 1.00 |
| Sodium Stearoyl Glutamate | 0.25 | 0.25 | 0.25 |
| Silica Dimethyl Silylate | 1.00 | 1.00 | 1.00 |
| Triacontanyl PVP | 1.00 | 1.00 | 1.00 |
| Parfum | as | as | as |
| | required | required | required |
| Glycerol | 8.60 | 8.60 | 8.60 |
| Sodium Hydroxide | as | as | as |
| | required | required | required |
| Stearyl Alcohol | 1.25 | 1.25 | 1.25 |
| Acrylates/C10-30 Alkyl Acrylate | 0.15 | 0.15 | 0.15 |
| Crosspolymer | | | |
| Xanthan Gum | 0.40 | 0.40 | 0.40 |
| Tetrasodium Iminodisuccinate | 0.75 | 0.75 | 0.75 |
| Sodium Carboxymethylcellulose | 0.50 | 0.50 | 0.50 |
| Alcohol Denat. | 5.00 | 5.00 | 5.00 |
| Trisodium EDTA | 0.20 | 0.20 | 0.20 |
| Aqua | to 100 | to 100 | to 100 |

| | |
|---|---|
| INCI | Ex. 7 |

| | | | |
|---|---|---|---|
| Encapsulated | Bis-Ethylhexyloxyphenol | 6.66 | 4.00 |
| anisotriazine | Methoxyphenyl Triazine | | |
| | Cellulose Acetate | | 2.66 |

| | |
|---|---|
| Butyl Methoxydibenzoylmethane | 4.75 |
| Homosalate | 9.00 |
| Ethylhexyl Salicylate | 4.75 |
| Ethylhexyl Triazone | 3.00 |
| Titanium Dioxide (nano) | 0.82 |
| Silica | 0.15 |
| Dimethicone | 0.04 |
| Phenylbenzimidazole Sulfonic Acid | 1.00 |
| Tocopheryl Acetate | 0.06 |
| Hydroxyacetophenone | 0.40 |
| Panthenol | 1.05 |
| Ethylhexylglycerol | 0.30 |
| C12-15 Alkyl Benzoate | 2.00 |
| Butylene Glycol Dicaprylate/Dicaprate | 2.00 |
| C18-38 Alkyl Hydroxystearoyl Stearate | 1.00 |
| Glyceryl Stearate | 1.00 |
| Sodium Stearoyl Glutamate | 0.25 |
| Silica Dimethyl Silylate | 1.00 |
| Triacontanyl PVP | 1.00 |
| Parfum | as required |
| Glycerol | 8.60 |
| Sodium Hydroxide | as required |
| Stearyl Alcohol | 1.25 |
| Acrylates/C10-30 Alkyl Acrylate Crosspolymer | 0.15 |
| Xanthan Gum | 0.40 |
| Alcohol Denat. | 5.00 |
| Trisodium EDTA | 0.20 |
| Aqua | to 100 |

1.-15. (canceled)

16. A cosmetic preparation, wherein the preparation comprises 2,4-bis{[4-(2-ethylhexyloxy)-2-hydroxy]-phenyl}-6-(4-methoxyphenyl)-1,3,5-triazine encapsulated with cellulose acetate.

17. The cosmetic preparation of claim 16, wherein the preparation further comprises one or more UV filters selected from 4-(tert-butyl)-4′-methoxydibenzoylmethane, salts of 2-phenylbenzimidazole-5-sulfonic acid, 2,4,6-tris[anilino-(p-carbo-2′-ethyl-1′-hexyloxy)]-1,3,5-triazine (INCI: Ethylhexyl Triazone), 2,4-bis{[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine), 4,4′-[[6-[[4-[[(1,1-dimethylethyl)amino]carbonyl]phenyl]amino]-1,3,5-triazine-2,4-diyl]diimino]bis,bis(2-ethylhexyl)benzoate (INCI: Diethylhexyl Butamido Triazone), 2,4,6-tribiphenyl-4-yl-1,3,5-triazine (INCI: Tris-Biphenyl Triazine), 2-ethylhexyl 2-hydroxybenzoate (INCI: Ethylhexyl Salicylate), 3,3,5-trimethylcyclohexyl 2-hydroxybenzoate (INCI: Homosalate), 2,2′-methylenebis(6-(2H-benzotriazol-2-yl)-4-(1,1,3,3-tetramethylbutyl)-phenol), 2-ethylhexyl 2-cyano-3,3-diphenylacrylate, 2-(2H-benzotriazol-2-yl)-4-methyl-6-[2-methyl-3-[1,3,3,3-tetramethyl-1-[(trimethylsilyl)oxy]-1-disiloxanyl]propyl]phenol (INCI: Drometrizole Trisiloxane), [(3Z)-3-[[4-[(Z)-[7,7-dimethyl-2-oxo-1-(sulfomethyl)-3-bicyclo[2.2.1]heptanylidene]methyl]phenyl]methylidene]-7,7-dimethyl-2-oxo-1-bicyclo[2.2.1]heptanyl]methanesulfonic acid (INCI: Terephthalylidene Dicamphor Sulfonic Acid), merocyanine, titanium dioxide, zinc oxide.

18. The cosmetic preparation of claim 16, wherein the preparation comprises 2,4-bis{[4-(2-ethylhexyloxy)-2-hydroxy]-phenyl}-6-(4-methoxyphenyl)-1,3,5-triazine encapsulated with cellulose acetate in an amount of from 1% to 7% by weight based on a total weight of the preparation.

19. The cosmetic preparation of claim 16, wherein a weight ratio of cellulose acetate to 2,4-bis{[4-(2-ethylhexyloxy)-2-hydroxy]-phenyl}-6-(4-methoxyphenyl)-1,3,5-triazine in the capsules is from 30:70 to 50:50.

20. The cosmetic preparation of claim 16, wherein the preparation is in the form of an emulsion.

21. The cosmetic preparation of claim 16, wherein the preparation further comprises one or more of glyceryl stearate citrate, cetearyl alcohol, sodium cetearyl sulfate, glyceryl stearate, cetearyl sulfosuccinate, sodium stearoyl glutamate, polyglyceryl-3-methylglucose distearate, polyglyceryl-3-methylglucose distearate, stearic acid, potassium cetyl phosphate, polyglyceryl-10 stearate (INCI Polyglyceryl-10 Stearate), polyglyceryl-2 caprate.

22. The cosmetic preparation of claim 16, wherein the preparation is free of polyethylene glycol, polyethylene glycol ethers and polyethylene glycol esters.

23. The cosmetic preparation of claim 16, wherein the preparation further comprises one or more of ethylhexylglycerol, propylene glycol, butylene glycol, 2-methylpropane-1,3-diol, 1,2-pentanediol, 1,2-hexanediol, 1,2-octanediol, and/or 1,2-decanediol.

24. The cosmetic preparation of claim 23, wherein the preparation comprises ethylhexylglycerol.

25. The cosmetic preparation of claim 16, wherein the preparation further comprises one or more of phenoxyethanol, 4-hydroxyacetophenone, ethanol.

26. The cosmetic preparation of claim 23, wherein the preparation further comprises one or more of phenoxyethanol, 4-hydroxyacetophenone, ethanol.

27. The cosmetic preparation of claim 24, wherein the preparation further comprises phenoxyethanol.

28. The cosmetic preparation of claim 16, wherein the preparation further comprise one or more perfumes selected from limonene, citral, linalool, alpha-isomethyl ionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methyl ionone, amyl C, butylphenyl methylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, ethylene brassylate, Evernia furfuracea extract, Evernia prunastri extract, farnesol, guaiac wood oil, geraniol, hexylcinnamal, hexyl salicylate, hydroxycitronellal, hydroxyisohexyl 3-cyclohexenecarboxaldehyde, lavender oil, lime oil, linalyl acetate, mandarin oil, menthyl PCA, methylheptenone, nutmeg oil, rosemary oil, sweet orange oil, terpineol, tonka bean oil, triethyl citrate, vanillin.

29. The cosmetic preparation of claim 16, wherein the preparation is free of parabens, methylisothiazolinone, chloromethylisothiazolinone, 3-iodo-2-propynyl butylcarbamate, DMDM hydantoin, 2-hydroxy-4-methoxybenzophenone, 2-ethylhexyl 4-methoxycinnamate, isoamyl 4-methoxycinnamate, 3-(4-methylbenzylidene)camphor.

30. The cosmetic preparation of claim 16, wherein the preparation further comprises one or both of silica dimethyl silylate and talc.

31. The cosmetic preparation of claim 16, wherein the preparation further comprises one or more oils selected from butylene glycol dicaprylate/dicaprate, phenethyl benzoate, C12-15 alkyl benzoate, dibutyl adipate, diisopropyl sebacate, dicaprylyl carbonate, di-C12-13 alkyl tartrate, butyloctyl salicylate, diethylhexyl syringylidenemalonate, hydrogenated castor oil dimerate, triheptanoin, C12-13 alkyl lactate, C16-17 alkyl benzoate, propylheptyl caprylate, caprylic/capric triglyceride, diethylhexyl 2,6-naphthalate, octyldodecanol, caprylic/capric triglyceride, ethylhexyl cocoate.

32. The cosmetic preparation of claim 16, wherein the preparation further comprises one or more compounds selected from alpha-lipoic acid, folic acid, phytoene, D-biotin, coenzyme Q10, alpha-glucosylrutin, carnitine, carnosine, glycyrrhetinic acid, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, glycerylglucose, (2-hydroxyethyl)urea, vitamin E and derivatives thereof, hyaluronic acid and/or salts thereof, licochalcone A.

33. The cosmetic preparation of claim 16, wherein preparation further comprises Acrylate/C10-30 Alkyl Acrylate Crosspolymer and/or triacontanyl PVP.

34. The cosmetic preparation of claim 16, wherein the preparation further comprises one or more of xanthan gum, tapioca starch, carboxymethyl cellulose.

35. A method facilitating the washability of a cosmetic preparation containing 2,4-bis{[4-(2-ethylhexyloxy)-2-hydroxy]-phenyl}-6-(4-methoxyphenyl)-1,3,5-triazine out of textiles contaminated with the preparation, wherein the process comprises encapsulating the 2,4-bis{[4-(2-ethylhexyloxy)-2-hydroxy]-phenyl}-6-(4-methoxyphenyl)-1,3,5-triazine present in the preparation with cellulose acetate.
